(19) United States
(12) Patent Application Publication
(10) Pub. No.: US 2019/0290240 A1
(43) Pub. Date: Sep. 26, 2019

(54) Medical Apparatus with Ultrasound Image, Tracking and Balloon Inflation

(71) Applicant: Gyrus ACMI, Inc. d.b.a. Olympus Surgical Technologies America, Southborough, MA (US)

(72) Inventor: Ming J. Cheng, W. Warwick, RI (US)

(73) Assignee: Gyrus ACMI, Inc. d.b.a. Olympus Surgical Technologies America

(21) Appl. No.: 15/933,982

(22) Filed: Mar. 23, 2018

(51) Int. Cl. A61B 8/08; A61M 29/02

(52) U.S. Cl. CPC A61B 8/0841; A61M 25/09; A61M 29/02

(57) ABSTRACT

Disclosed herein is a medical apparatus. The medical apparatus includes an ultrasound probe, a handpiece, and a pedal member. The ultrasound probe is configured to be externally positioned on a facial area of a patient. The handpiece has a guidewire and a balloon catheter. The balloon catheter is configured to be in fluid communication with a fluid source. The pedal member is configured to operably manipulate the fluid source. The ultrasound probe is configured to position the balloon catheter to a nasal ostium of the patient for dilation of the nasal ostium through the guidewire.

### BACKGROUND

### Field of the Invention

The invention relates to a medical apparatus and more specifically relates to a medical apparatus with ultrasound image, tracking and balloon inflation.

### Brief Description of Prior Developments

Balloon sinuplasty relates to a sinus surgery procedure for the treatment of blocked sinuses. Balloon sinuplasty surgery generally includes using a balloon over a wire catheter to dilate sinus passageways. During the procedure, the balloon is inflated to provide for dilating the sinus openings, widening the walls of the sinus passageway and restoring normal drainage.

According to various conventional procedures, the physician inserts a guide catheter through the nostril and near the sinus opening under endoscopic visualization. A flexible guide wire is inserted into the inflamed sinus. According to various exemplary configurations, many guide wires have a light which can be seen through the skin to help the physician with correct placement of the guide wire. For example, some device configurations manufactured by Acclarent, Inc., use an optical fiber LED light integrated onto the guide wire. After placement of the guide wire, a balloon catheter is advanced over the guide wire so that it can be positioned at the inflamed sinus. The balloon is then inflated to expand the sinus. The goal of the procedure is that the sinus will remain open or widened after the balloon is deflated and removed.

Accordingly, there is a need to provide improved and reliable medical device configurations.

### SUMMARY

In accordance with one aspect of the invention, a medical apparatus is disclosed. The medical apparatus includes an ultrasound probe, a handpiece, and a pedal member. The ultrasound probe is configured to be externally positioned on a facial area of a patient. The handpiece has a guidewire and a balloon catheter. The balloon catheter is configured to be in fluid communication with a fluid source. The pedal member is configured to operably manipulate the fluid source. The ultrasound probe is configured to position the balloon catheter to a nasal ostium of the patient for dilation of the nasal ostium through the guidewire.

In accordance with one aspect of the invention, a medical apparatus is disclosed. The medical apparatus includes an ultrasound probe, a handpiece, and a pedal member. The ultrasound probe is configured to be externally positioned on a facial area of a patient. The handpiece has a distal tip and a balloon catheter. The balloon catheter is configured to be in fluid communication with a fluid source. The pedal member is configured to operably manage the fluid source. The ultrasound probe is configured to guide the distal tip of the handpiece through a nasal cavity to a nasal ostium of the patient through a monitoring screen.

### BRIEF DESCRIPTION OF THE DRAWINGS

The foregoing aspects and other features of the invention are explained in the following description, taken in connection with the accompanying drawings, wherein:

FIG. 1 is a perspective view of a medical apparatus incorporating features of the invention;

FIG. 2 is a perspective view of a handpiece (partially disassembled) of the medical apparatus shown in FIG. 1;

FIG. 3 is another perspective view of the handpiece (partially disassembled) of the medical apparatus shown in FIG. 1;

FIG. 4 is a section view of the handpiece shown in FIGS. 2, 3;

FIG. 5 is a perspective view of a pedal assembly of the medical apparatus shown in FIG. 1; and

FIG. 6 is another section view of the handpiece shown in FIGS. 2, 3.

### DETAILED DESCRIPTION

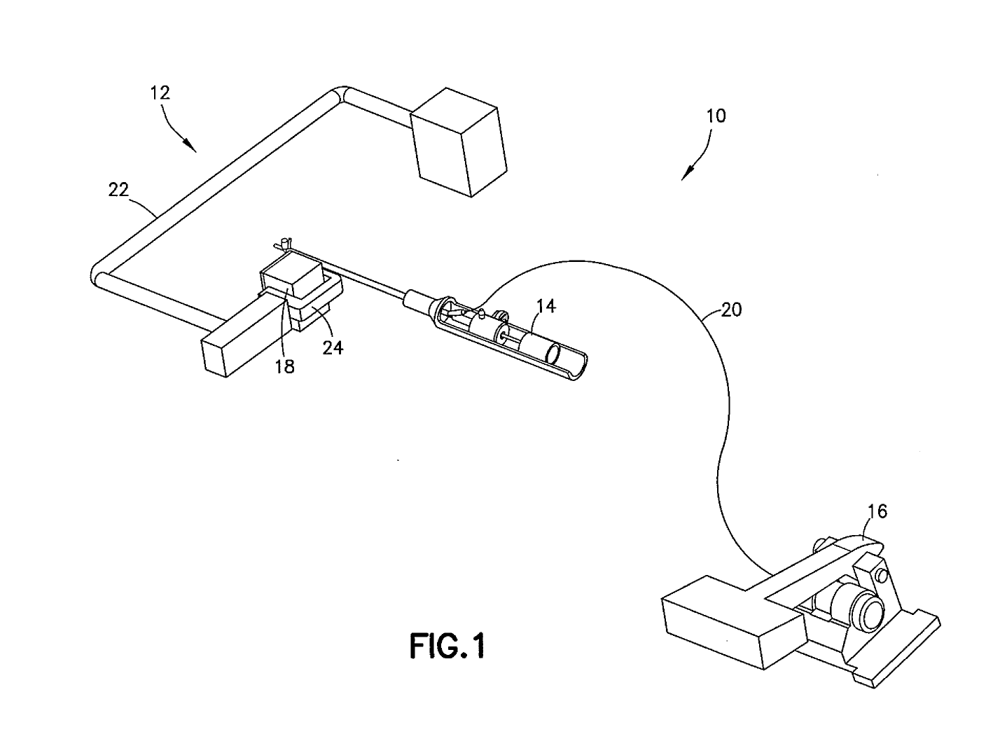

Referring to FIG. 1, there is shown a perspective view of a medical apparatus 10 (which may be an interventional sinus endoscope [ISE] apparatus, for example) incorporating features of the invention. Although the invention will be described with reference to the exemplary embodiments shown in the drawings, it should be understood that the invention can be embodied in many alternate forms of embodiments. In addition, any suitable size, shape or type of elements or materials could be used.

The medical apparatus 10 comprises a head setting 12, a handpiece 14, and a pedal assembly 16. The head setting (or head set) 12 comprises an ultrasound probe 18 configured to provide ultrasound imaging to assist in positioning and location of the handpiece 14 during medical procedures. The handpiece 14 is connected to the pedal assembly 16 by a balloon tube 20.

The head setting 12 comprises a frame 22 and a receiving area 24. The receiving area 24 is configured to removably receive the ultrasound probe 18. The head setting 12 is configured to allow a user to fix a position of the ultrasound probe 18 when a suitable patient ultrasound image is found (during medical procedures such as a balloon sinuplasty procedure, for example).

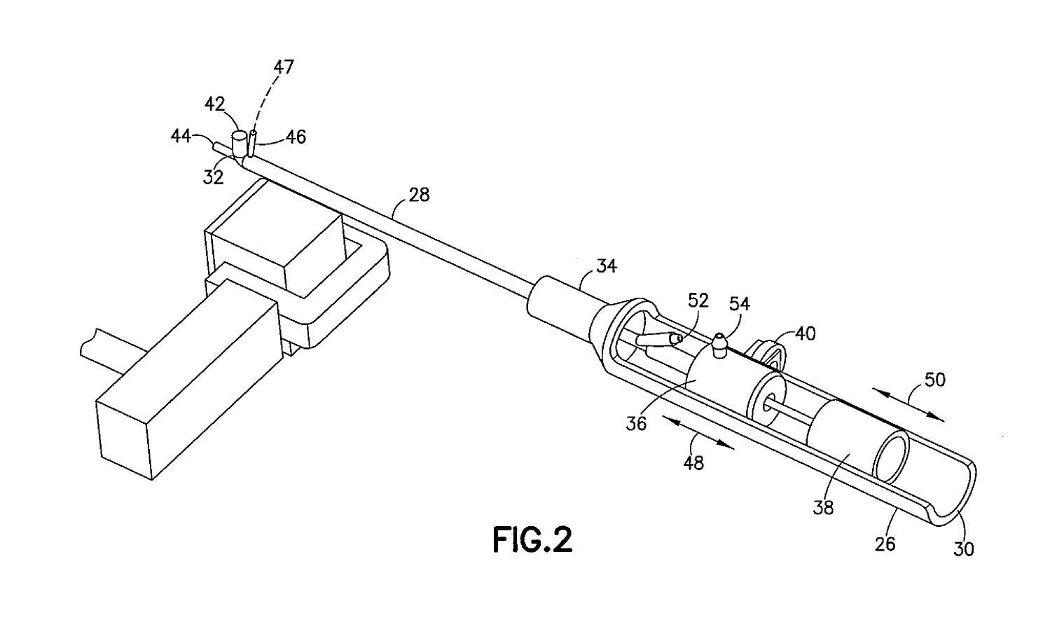

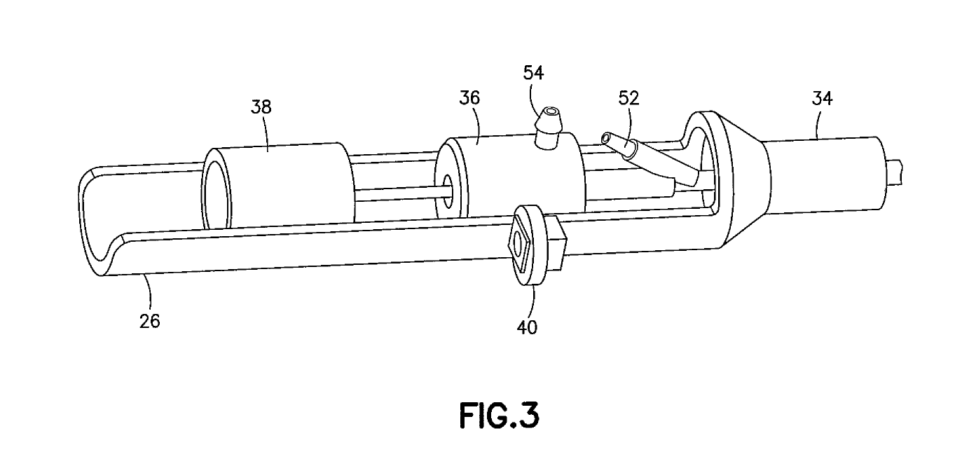

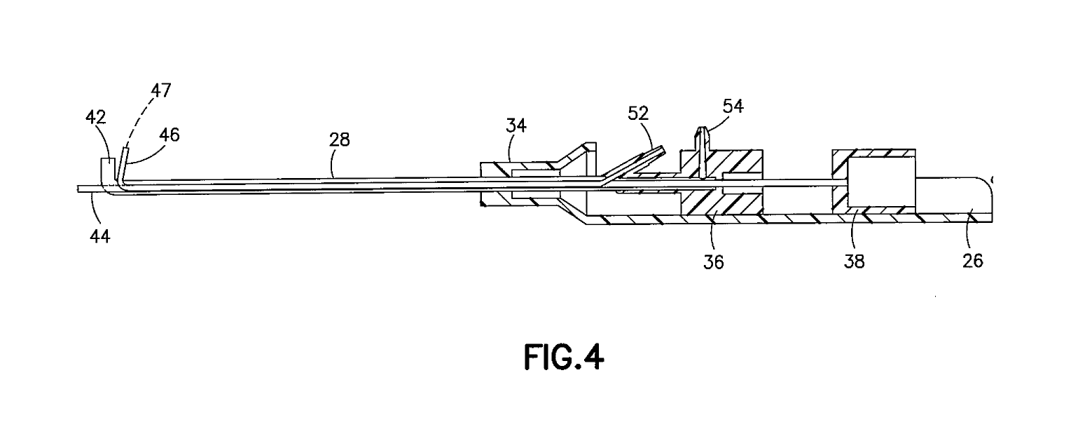

The handpiece 14 (also shown in FIGS. 2-4), which may be a sinuplasty handpiece, comprises a housing 26 and a tube section 28 extending between a proximal end 30 of the handpiece and a distal tip 32 of the handpiece such that the tube section 28 is connected to the housing 26 at a connecting area 34 of the housing 26. Within the housing 26 there is provided a catheter adapter (or catheter slider) 36 and a guide wire adapter (or guide wire slider) 38. The catheter adapter 36 is between the proximal end 30 and the connecting area 34. The guide wire adapter 38 is between the catheter adapter 36 and the proximal end 34. Additionally, a catheter adapter locator 40 is connected to the housing 26 adjacent the catheter adapter 36.

The handpiece 12 is configured to have a pipe portion 42 at the distal tip 32 with a guide wire 44 and a catheter 46 with a balloon 47 extending therefrom. The guide wire 44 and the catheter 46 (with balloon 47) are connected to the catheter adapter 36 and the guide wire adapter 38, respectively, through the tube section 28. The catheter adapter 36 and the guide wire adapter 38 are configured to slide within the housing 26 (see arrows 48, 50) when the handpiece is in use. Additionally, various exemplary embodiments include a balloon inflation connector 52 (for connection with the balloon tube 20)) between the connecting area 34 and the catheter adaptor and a suction/flush connector 54 at the catheter adaptor 36.

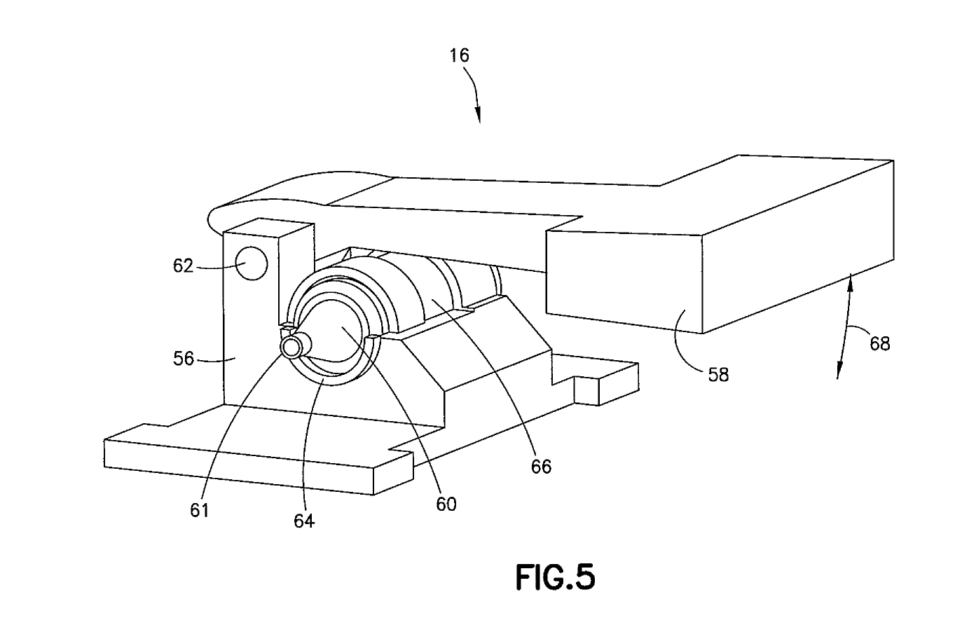

Referring now also to FIG. 5, the pedal assembly 16 comprises a base section 56, a pedal member 58 and a fluid source 60. The base section 56 comprises a pedal pivot pin 62 and a container receiving area 64. The fluid source 60 may be a flexible container (such as a rubber container, for example) comprising a compressed area 66. The flexible container 60 is configured to hold fluid (such as saline, for example) and connect to the balloon tube 20 at tube connector 61 (such that the balloon catheter 46 is in fluid communication with the fluid source 60 through the balloon inflation connector 52). The flexible container 60 is received in the container receiving area 64 such that the compressed area 66 is aligned with the pedal member 58. The pedal member 58 (which may be a foot pedal, for example) is configured to pivot about pivot pin 62 (see arrow 68) and make contact with the compressed area 66 of the flexible container 60.

While various exemplary embodiments of the invention have been described in connection with an apparatus, it should be noted that the various exemplary embodiments of the invention are not necessarily so limited and that a method of using the apparatus is also disclosed. For example, according to various exemplary embodiments of the invention, the medical apparatus may be used in a sinuplasty surgery procedure as follows: first a surgeon uses the ultrasound probe 18 to find patient best image and then fix it by the head set 12. Next, using the guide wire 44 based upon Ultrasound technology and reaching out the right target location. Next, the catheter 46 is advanced along the guide wire 44 to the inflammation section. Next, the balloon 47 is inflated to open blocked passageways and restructure the sinus passageway. Next, the balloon 47 is deflated. Next, the mucus in the sinus can be pumped out and flushed to drain the passageway without pulling out the guide wire 44. Next, the ultrasound probe 18 can be used to check if any mucus is still in the passageway.

It should be noted that the particular order of the actions/procedures above do not necessarily imply that there is a required or preferred order for the actions/procedures and the order and arrangement of the actions/procedures may be varied. Furthermore, it may be possible for some actions/procedures to be omitted.

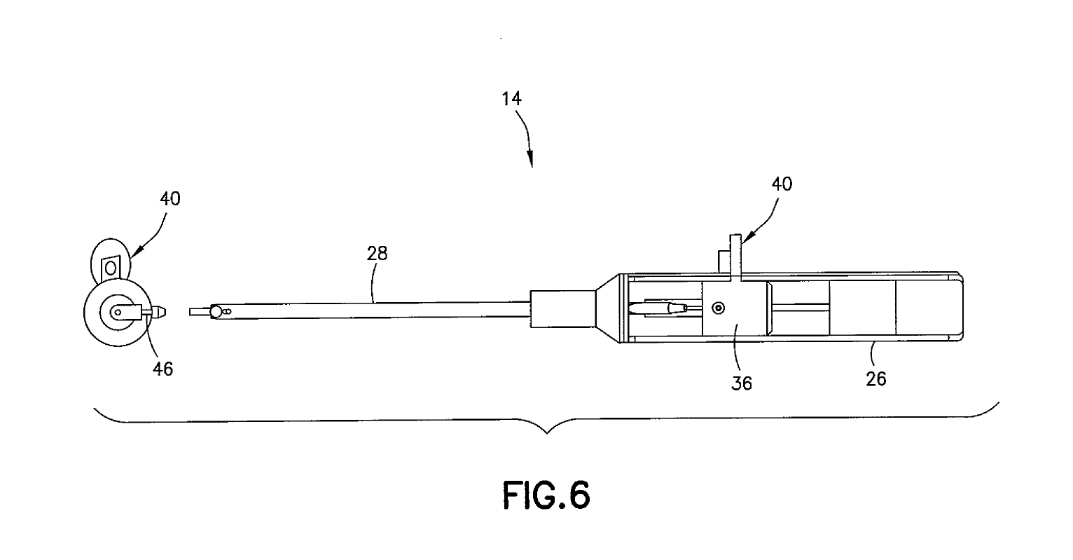

Referring now also to FIG. 6, according to various exemplary embodiments, the balloon catheter 46 seats on top of the catheter slider 36 and may go along with the guide wire 44 when guide wire slider 38 is activated, therefore the catheter adapter locator 40 is included to provide a firm stop to ensure position accuracy between the guide wire 44 and the balloon catheter 46. This configuration is similar to a cam and follower mechanism by adjusting the friction force between cam and follower. While various exemplary embodiments of the invention have been described in connection with the catheter adapter locator 40 in direct contact with the catheter adapter 36, one skilled in the art will appreciate that the various exemplary embodiments are not necessarily so limited and that alternate embodiments may comprise configurations where the catheter adapter locator 40 is not in direct contact with the catheter adapter 36. For example, in some embodiments the catheter adapter locator 40 may be connected to the catheter adapter 36 through an intermediary member that transfers movement from the catheter adapter locator 40 to the catheter adapter 36, in other embodiments the catheter adapter locator 40 may act as an electronic switch connected to another member within the handpiece 14 which physically contacts the catheter adapter 36. Additionally, in other alternate embodiments any suitable configuration between the catheter adapter locator 40 and the catheter adapter 36 may be provided.

The pedal assembly 16 is configured such that the pedal member 58 works as leverage by exertion of force on the pedal member 58 to compress the flexible container 60 which contains saline to generate a required pressure (such as about 150-180 psi, for example). With this configuration, the surgeon can easily just push the pedal member 58 to finish balloon inflation and release the pedal to deflate it.

Various exemplary embodiments of the invention provide for using ultrasound technology to guide and track the guide wire movement and also to verify if there is any mucus still left in sinus cavity. Additionally, since an ultrasound probe is used, the guide wire can be even smaller, such as about 1.5 mm or less in diameter (whereas conventional guide wires are about 2 mm or larger in diameter). This reduction in size means that it will be a more comfortable when being inserted into a patient sinus canal, also this will increase the gap between guide wire and balloon catheter so it greatly helps suction process, and further with this configuration the guide wire does not need to be pulled out before suction. Furthermore, various exemplary embodiments provide a cost savings as the balloon inflation and deflation configuration (using the pedal assembly 16, for example) eliminates the need for a person who take care of the balloon inflation/deflation during a conventional sinuplasty process.

Technical effects of any one or more of the exemplary embodiments provide significant advantages over conventional configurations by providing improved configurations. For example current configurations, such as provided by Acclarent, Inc., uses an optical fiber LED light integrated onto the guide wire to ensure the guide wire is in a right spot or cavity during its advancing thru the ostium, so it inevitably make its guide wire bigger to accommodate this feature which conversely slows down the suction process because the gap between guide wire and catheter (sliding over the guide wire) is too small to draw out mucus, so the surgeon has to withdraw the guide wire before sucking operations. Additionally, with the current configurations there are two people required to have to be involved in the sinuplasty procedure: one is the surgeon holding the instrument and scope and concurrently a nurse is needed to inflation and deflation of the balloon for the balloon sinuplasty. Furthermore, in conventional configurations after the sinuplasty operation there is no way for surgeons to confirm that mucus is fully drawing off or not.

Various exemplary embodiments of the invention provide for using an ultrasound probe instead of a scope, to provide advantages such as: using ultrasound probe not only presents 2D image but also can track the guide wire in real time (such as on a monitoring screen [not shown] connected to the ultrasound probe) to confirm the correct site, The ultrasound probe can ensure that no mucus is left on the site, and the guide wire is smaller in size and simple in design and has enough flexibility to move around in the cavity.

Below are provided further descriptions of various non-limiting, exemplary embodiments. The below-described exemplary embodiments may be practiced in conjunction with one or more other aspects or exemplary embodiments. That is, the exemplary embodiments of the invention, such as those described immediately below, may be implemented, practiced or utilized in any combination (e.g., any combination that is suitable, practicable and/or feasible) and are not limited only to those combinations described herein and/or included in the appended claims.

In one exemplary embodiment, a medical apparatus comprising: an ultrasound probe configured to be externally positioned on a facial area of a patient; a handpiece having a guidewire and a balloon catheter, the balloon catheter configured to be in fluid communication with a fluid source; a pedal member configured to operably manipulate the fluid source; wherein the ultrasound probe is configured to position the balloon catheter to a nasal ostium of the patient for dilation of the nasal ostium through the guidewire.

A medical apparatus as above, wherein the pedal member comprises a foot pedal member.

A medical apparatus as above, wherein the pedal member is configured to operate, control, and/or manage the fluid source.

A medical apparatus as above, wherein the fluid source comprises a flexible container.

A medical apparatus as above, wherein the handpiece comprises a distal tip.

A medical apparatus as above, wherein the ultrasound probe is configured to guide the distal tip of the handpiece through a nasal cavity to a nasal ostium of the patient through a monitoring screen.

A medical apparatus as above, wherein the handpiece and the pedal member are configured to be simultaneously operated by a single person.

A medical apparatus as above, wherein the handpiece comprises a catheter adapter and a guidewire adapter within a housing of the handpiece.

A medical apparatus as above, wherein the handpiece further comprises a catheter adapter locator connected to the housing, wherein the catheter adapter locator is in contact with the catheter adapter.

In another exemplary embodiment, a medical apparatus comprising: an ultrasound probe configured to be externally positioned on a facial area of a patient; a handpiece having a distal tip and a balloon catheter, the balloon catheter configured to be in fluid communication with a fluid source; a pedal member configured to operably manage the fluid source; wherein the ultrasound probe is configured to guide the distal tip of the handpiece through a nasal cavity to a nasal ostium of the patient through a monitoring screen.

A medical apparatus as above, wherein the pedal member comprises a foot pedal member.

A medical apparatus as above, wherein the pedal member is configured to operate, control, and/or manage the fluid source.

A medical apparatus as above, wherein the fluid source comprises a flexible container.

A medical apparatus as above, wherein the handpiece comprises a guidewire.

A medical apparatus as above, wherein the ultrasound probe is configured to position the balloon catheter to a nasal ostium of the patient for dilation of the nasal ostium through the guidewire.

A medical apparatus as above, wherein the handpiece and the pedal member are configured to be simultaneously operated by a single person.

A medical apparatus as above, wherein the handpiece comprises a catheter adapter and a guidewire adapter within a housing of the handpiece.

A medical apparatus as above, wherein the handpiece further comprises a catheter adapter locator connected to the housing, wherein the catheter adapter locator is in contact with the catheter adapter.

It should be understood that components of the invention can be operationally coupled or connected and that any number or combination of intervening elements can exist (including no intervening elements). The connections can be direct or indirect and additionally there can merely be a functional relationship between components.

It should be understood that the foregoing description is only illustrative of the invention. Various alternatives and modifications can be devised by those skilled in the art without departing from the invention. Accordingly, the invention is intended to embrace all such alternatives, modifications and variances which fall within the scope of the appended claims.

What is claimed is:

1. A medical apparatus comprising:
an ultrasound probe configured to be externally positioned on a facial area of a patient;
a handpiece having a guidewire and a balloon catheter, the balloon catheter configured to be in fluid communication with a fluid source;
a pedal member configured to operably manipulate the fluid source;
wherein the ultrasound probe is configured to position the balloon catheter to a nasal ostium of the patient for dilation of the nasal ostium through the guidewire.

2. The medical apparatus of claim 1 wherein the pedal member comprises a foot pedal member.

3. The medical apparatus of claim 1 wherein the pedal member is configured to operate, control, and/or manage the fluid source.

4. The medical apparatus of claim 1 wherein the fluid source comprises a flexible container.

5. The medical apparatus of claim 1 wherein the handpiece comprises a distal tip.

6. The medical apparatus of claim 5 wherein the ultrasound probe is configured to guide the distal tip of the handpiece through a nasal cavity to a nasal ostium of the patient through a monitoring screen.

7. The medical apparatus of claim 1 wherein the handpiece and the pedal member are configured to be simultaneously operated by a single person.

8. The medical apparatus of claim 1 wherein the handpiece comprises a catheter adapter and a guidewire adapter within a housing of the handpiece.

9. The medical apparatus of claim 8 wherein the handpiece further comprises a catheter adapter locator connected to the housing, wherein the catheter adapter locator is in contact with the catheter adapter.

10. A medical apparatus comprising:
an ultrasound probe configured to be externally positioned on a facial area of a patient;
a handpiece having a distal tip and a balloon catheter, the balloon catheter configured to be in fluid communication with a fluid source;
a pedal member configured to operably manage the fluid source;
wherein the ultrasound probe is configured to guide the distal tip of the handpiece through a nasal cavity to a nasal ostium of the patient through a monitoring screen.

11. The medical apparatus of claim 10 wherein the pedal member comprises a foot pedal member.

12. The medical apparatus of claim 10 wherein the pedal member is configured to operate, control, and/or manage the fluid source.

13. The medical apparatus of claim 10 wherein the fluid source comprises a flexible container.

14. The medical apparatus of claim 10 wherein the handpiece comprises a guidewire.

15. The medical apparatus of claim 14 wherein the ultrasound probe is configured to position the balloon catheter to a nasal ostium of the patient for dilation of the nasal ostium through the guidewire.

16. The medical apparatus of claim 10 wherein the handpiece and the pedal member are configured to be simultaneously operated by a single person.

17. The medical apparatus of claim 10 wherein the handpiece comprises a catheter adapter and a guidewire adapter within a housing of the handpiece.

18. The medical apparatus of claim 17 wherein the handpiece further comprises a catheter adapter locator connected to the housing, wherein the catheter adapter locator is in contact with the catheter adapter.
